(19) United States
(12) Patent Application Publication
(10) Pub. No.: US 2022/0166508 A1
(43) Pub. Date: May 26, 2022

(54) BIO-INFORMATION TRANSMITTER, BIO-INFORMATION RECEIVER, AND BIO-INFORMATION COMMUNICATION SYSTEM

(71) Applicant: KOOKMIN UNIVERSITY INDUSTRY ACADEMY COOPERATION FOUNDATION, Seoul (KR)

(72) Inventors: Yeong Min JANG, Seoul (KR); AHMED MD FAISAL, Seoul (KR)

(21) Appl. No.: 17/535,058

(22) Filed: Nov. 24, 2021

(30) Foreign Application Priority Data
Nov. 24, 2020 (KR) 10-2020-0159239

(51) Int. Cl. H04B 10/116; G08C 23/04; H04N 5/235; H04B 10/50; A61B 5/00

(52) U.S. Cl. CPC H04B 10/116; G08C 23/04; A61B 5/14552; H04B 10/502; A61B 5/0013; H04N 5/2353

(57) ABSTRACT

An optical communication system is disclosed. The present system includes a light emitting element array for optically transmitting bio-data of a person to be diagnosed, and an optical receiver for recognizing color of the light emitting element array by using an image sensor to acquire the bio-data of the person to be diagnosed. Accordingly, an untact diagnostic test may be performed.

### CROSS-REFERENCE TO RELATED APPLICATIONS

The present application claims priority under 35 U.S.C 119(a) to Korean Patent Application No. 10-2020-0159239, filed on Nov. 24, 2020, which is incorporated herein by reference in its entirety.

### BACKGROUND

### 1. Technical Field

The present disclosure relates to a transmitter and a receiver respectively for transmitting and receiving bio-information by using light, and an optical communication system to which the transmitter and receiver are applied.

### 2. Related Art

Visible light communication (VLC) that is a representative light communication convergence technology is a technology for wireless communication by loading information on light of a light source and is a technology of the related art that receives light from a light source through a photo diode (PD), detects digital data of 1 or 0 depending on turn-on and turn-off of the light source, and transmits information according to a combination thereof.

In the related art, there has been proposed a visible light communication system in which a plurality of light emitting diodes (LEDs) are imaged by using a camera instead of a photodiode and data depending on turn-on and turn-off of the LEDs obtained for each frame of the camera is extracted. In this way, visible light communication using a camera is also called an optical camera communication (OCC) system in that a camera instead of a photodiode is used as an optical receiver, and An IEEE 802.15.7a study group is working on standardization.

Meanwhile, due to a high interest in health, health diagnosis has been actively conducted. One of the most popular diagnostic tests is probably the diagnostic test that checks a heart rate and oxygen saturation (SpO₂).

The diagnostic test is generally conducted face-to-face between a diagnostician and a person to be diagnosed. However, there is a clear tendency to avoid face-to-face diagnosis due to the recent COVID-19, and in order to reduce labor costs, a method of high test efficiency is required while conducting a diagnostic test on the person to be diagnosed by using a diagnostic test device without a diagnostician.

Meanwhile, the above information is only presented as background information to help understanding of the present disclosure. No decision has been made, no claim is made as to whether any of the descriptions is applicable to the present disclosure as the related art.

An example of related art is Korean Publication No. 10-2013-0067489 (published date: Jun. 25, 2013).

### SUMMARY

The present disclosure provides a method of acquiring bio-data of a person to be diagnosed in an untact manner.

The present disclosure further provides a method of acquiring accurate bio-data while effectively monitoring a person to be diagnosed in real time.

The present disclosure further provides a method of transmitting data by using a light emitting element array and acquiring the corresponding data by using an optical camera including an image sensor.

The present disclosure further provides an optical communication system encrypting bio-data for each user and transmitting and receiving the data.

Technical problems to be achieved in the present disclosure are not limited to the technical problems described above, and other technical problems not described will be clearly understood by those skilled in the art to which the present disclosure belongs from the description below.

According to an aspect of the present disclosure, there is provided a bio-information transmitting device including at least one biometric sensor configured to acquire a plurality of bio-data, a light emitting element array, and a transmission controller configured to map a light emitting element group included in the light emitting element array for each type of the bio-data, configured to encode the acquired bio-data into RGB-based color data, and configured to output the encoded color data through the light emitting element group.

Here, at least part of the light emitting element group may be configured to turn off one light emitting element included in the light emitting element group.

According to another aspect of the present disclosure, there is provided a bio-information receiving device including an optical camera configured to image a light emitting element array in an image range, and a reception controller configured to recognize the light emitting element array and configured to recognize an arrangement direction of light emitting elements included in the light emitting element array based on an arrangement direction estimation model which is previously stored, based on one or more light emitting elements which are turned off.

The reception controller may be configured to decode color data of a light emitting element group mapped for each type of a plurality of bio-data into corresponding bio-data to acquire decoded bio-data.

According to another aspect of the present disclosure, there is provided a bio-information communication system including a transmitter, and a receiver.

The transmitter may include at least one biometric sensor configured to acquire a plurality of bio-data, a light emitting element array, and a transmission controller configured to map a light emitting element group included in the light emitting element array for each type of the bio-data, configured to encode the acquired bio-data into RGB-based color data, and configured to output the encoded color data through the light emitting element group.

At least part of the light emitting element group may be configured to turn off one light emitting element included in the light emitting element group.

The receiver may include an optical camera configured to image a light emitting element array in an image range, and a reception controller configured to recognize the light emitting element array and configured to recognize an arrangement direction of light emitting elements included in the light emitting element array based on an arrangement direction estimation model which is previously stored, based on one or more light emitting elements which are turned off.

The reception controller may be configured to decode color data of a light emitting element group mapped for each type of a plurality of bio-data into corresponding bio-data to acquire decoded bio-data.

Solutions to the technical problems to be achieved in the present disclosure are not limited to the solutions described above, and other solutions not described will be understood by those skilled in the art to which the present disclosure belongs from the description below.

According to various embodiments of the present disclosure, accurate bio-data may be measured while an untact diagnostic test is performed, a diagnostic test may be effectively performed even without a diagnostician, bio-data for each user is encrypted by a unique key to be transmitted and received, and thus, personal information may be effectively protected.

### BRIEF DESCRIPTION OF THE DRAWINGS

Various embodiments of the present disclosure will become more apparent in view of the attached drawings and accompanying detailed description, in which:

FIG. 1 is a view for schematically illustrating an optical communication system according to an embodiment of the present disclosure;

FIG. 2 is a system block diagram illustrating a configuration of an optical communication system according to the embodiment of the present disclosure;

FIG. 3 is a diagram illustrating a position in which a light emitting element for intentionally disconnecting power to recognize an arrangement direction of a light emitting element array according to an embodiment of the present disclosure may be arranged;

FIG. 4 illustrates diagrams for a process of recognizing rotation of the light emitting element array and decoding color data, according to an embodiment of the present disclosure;

(a) and (b) of FIG. 5 are views illustrating a method of effectively recognizing color data output from a light emitting element by adjusting a light exposure time of an optical camera, according to an embodiment of the present disclosure;

FIG. 6 illustrates diagrams for examples of values of bio-data corresponding to the number of samples, according to an embodiment of the present disclosure;

FIG. 7 is a graph illustrating a relationship between normalized color intensity and a bit error rate (BER); and

FIG. 8 is a sequence diagram illustrating a communication method of an optical communication system according to an embodiment of the present disclosure.

### DETAILED DESCRIPTION OF THE EMBODIMENTS

Hereinafter, example embodiments of the present disclosure will be described with reference to the accompanying drawings. The present disclosure may be implemented in a variety of different forms and should not be construed as being limited to the embodiments described herein. Relative sizes of components, layers, and regions in the drawings may be exaggerated for the sake of clear description.

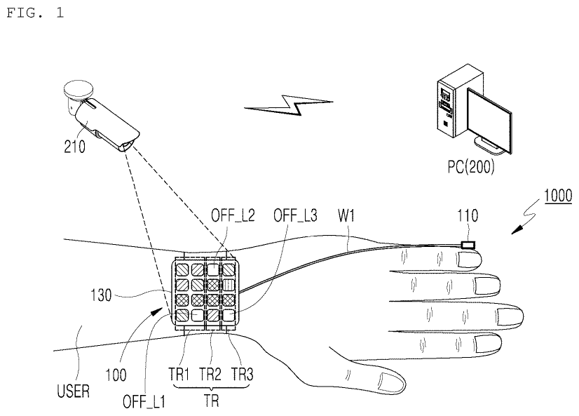

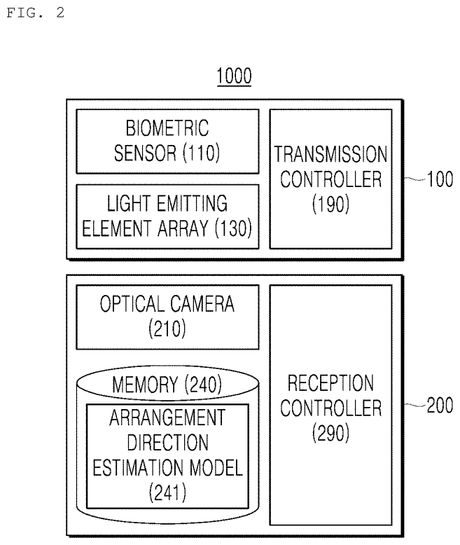

FIG. 1 is a view schematically illustrating an optical communication system 1000 according to an embodiment of the present disclosure.

The optical communication system 1000 may transmit and receive optical data by using the interaction between materials and light and include a transmitter 100 and a receiver 200.

The transmitter 100 may be located around a user's wrist, and the transmitter 100 may output RGB color data to the outside through a light emitting element array 130 including a plurality of light emitting elements. Here, the light emitting element array 130 may output color data as an optical signal, and the light emitting elements may each be implemented by an RGB-based light emitting diode (LED).

The transmitter 100 may optically output (transmit) various data and acquire a plurality of bio-data of a user USER through a biometric sensor 110 and transmit the plurality of acquired bio-data through a plurality of light emitting element groups TR1 to TR3.

Here, the transmitter 100 may map the light emitting element groups TR1 to TR3 for each type of measured bio-data. The light emitting element groups TR1 to TR3 may output color data related to the mapped bio-data.

Here, at least some (all groups in FIG. 1 are included) of the light emitting element groups TR1 to TR3 may include off-light emitting elements OFF_L1 to OFF_L3 in the groups, and each of the off-light emitting elements OFF_L1 to OFF_L3 may be used to distinguish data and may be used to recognize an arrangement direction of the light emitting elements (or the light emitting element array 130) when the receiver 200 recognizes the light emitting element array 130. That is, the receiver 200 may recognize whether the light emitting element array 130 is offside or rotates through the corresponding off-light emitting elements OFF_L1 to OFF_L3.

The biometric sensor 110 may include an oximeter sensor and may measure infrared (IR) data, beats per minute (BPM) data, oxygen saturation (SpO₂) data, and so on. Alternatively, the biometric sensor 110 may measure or calculate photoplethysmogram (PPG) data by using infrared rays. The biometric sensor 110 may be arranged at a finger end of the user USER. Here, the IR data refers to biometric information collected by using infrared rays, and the BPM data refers to data related to heart rates.

In an optional embodiment, the transmitter 100 may be provided separately from the biometric sensor 110 and communicate wirelessly.

The receiver 200 may recognize an arrangement direction of the light emitting element array 130 and color data of the light emitting elements included in the light emitting element array 130 by using the optical camera 210 including an image sensor, and then may decode the recognized color data to acquire bio-data.

In an optional embodiment, the receiver 200 may communicate with the optical camera 210 wirelessly or by wire and may be implemented by a server, a smartphone, a tablet device, and so on other than a personal computer (PC).

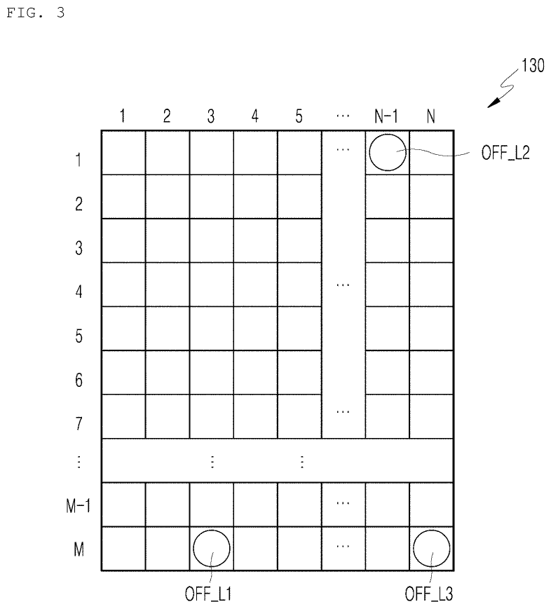

FIG. 2 is a system block diagram illustrating a configuration of the optical communication system 1000 according to the embodiment of the present disclosure. The optical communication system 1000 may include the transmitter 100 and the receiver 200.

First, the transmitter 100 may include the biometric sensor 110, the light emitting element array 130, and a transmission controller 190. In addition, the receiver 200 may include the optical camera 210, a memory 240 storing an arrangement direction estimation model 241 for estimating an arrangement direction of the light emitting element array 130, and a reception controller 290. The components illustrated in FIG. 2 are not essential for implementing the optical communication system 1000, and the optical communication system 1000 described in the present specification may include more or fewer components than the components listed above.

First, a configuration of the transmitter 100 will be described. The biometric sensor 110 may acquire a plurality of bio-data and include an oximeter sensor and include sensors for detecting various bio-data.

The light emitting element array 130 may be implemented in the form of a bundle in which a plurality of light emitting elements are arranged in an M×N matrix (M and N are natural numbers greater than or equal to 4) including a 4×4 matrix. The light emitting element array 130 may represent three types of bio-data but may also output more than three types of bio-data when a size thereof exceeds the 4×4 matrix.

The transmission controller 190 may map the light emitting element group included in the light emitting element array 130 for each type of bio-data. For example, the bio-data may include infrared (IR) data, beats per minute (BPM) data, and oxygen saturation (SpO₂) data. Referring to FIG. 2 together with FIG. 1, the transmission controller 190 may map first bio-data (for example, IR data) to the first light emitting element group TR1 and map second bio-data (for example, BPM data) to the second light emitting element group TR2 and map third bio-data (for example, SpO₂) to the third light emitting element group TR3.

The transmission controller 190 may encode the acquired bio-data into RGB-based color data and output the encoded color data through the light emitting element group. The color encoding method may use color intensity modulation (CIM) for encoding/decoding data according to color intensity.

The transmission controller 190 may control the light emitting elements included at least some of the light emitting element groups to be turned off. In one embodiment, the light emitting elements may not be arranged at all in the corresponding position instead of turning off the light emitting elements.

When the light emitting element array 130 is recognized by the receiver 200, the light emitting element to be turned off may help decisively to determine an arrangement direction of the light emitting element array 130 and also help decisively to distinguish bio-data and also help to acquire individual bio-data.

Next, the receiver 200 may include the optical camera 210 including an image sensor having a light receiving module, and the optical camera 210 may image the light emitting element array 130 in an image range. The optical camera 210 may be arrange between 1 meter and 3 meters, but the embodiment is not limited thereto.

The memory 240 may store various types of information and store the arrangement direction estimation model 241 which is previously trained. The arrangement direction estimation model 241 which is previously trained may be trained based on supervised learning.

First, the arrangement direction estimation model 241 may recognize the light emitting element array 130. In addition, the arrangement direction estimation model 241 may estimate an arrangement direction of the light emitting element array 130 based on the light emitting element that is turned off.

When the arrangement direction estimation model 241 receives information on one or more light emitting element arrays prepared in advance and arrangement information of the light emitting element that is turned off and included in a light emitting element group to be mapped to bio-data, the arrangement direction estimation model 241 may be trained to estimate the number of bio-data and the arrangement direction of the light emitting element included in the light emitting element array.

In addition, the arrangement direction estimation model 241 may estimate colors of the light emitting elements based on a label. That is, the arrangement direction estimation model 241 may be trained to estimate encoded color data included in each of the light emitting element groups when receiving the encoded color data of each of the light emitting elements included in one or more of the light emitting element arrays 130 prepared in advance.

Because the arrangement direction estimation model 241 according to the embodiment of the present disclosure may be generated by using a neural network algorithm, blurriness and distortion of images may be reduced through processes such as filtering, resampling, and smoothing used by existing technologies.

In this way, the reception controller 290 may recognize the light emitting element array 130 and may recognize an arrangement direction of a light emitting elements included in a light emitting element array based on one or more light emitting elements that are turned off by using the arrangement direction estimation model 241 which is previously stored.

FIG. 3 is a diagram illustrating a position in which a light emitting element for intentionally disconnect power to recognize an arrangement direction of a light emitting element array according to an example embodiment of the present disclosure. It is assumed that the light emitting element array 130 is composed of M rows and N columns, and M and N may be natural numbers of 4 or more.

If there are three types of bio-data, the M×N light emitting element array 130 may include three off-light emitting elements OFF_L1 to OFF_L3.

In an optional embodiment, the off-light emitting element that is turned off may be formed only between the bio-data and the bio-data (in this case, there may be two light emitting elements that are turned off). In addition, if only color data encoding and bio-data decoding may be performed, three or more types of bio-data may be further included in the light emitting element array 130.

Here, a position of the light emitting element that is turned off for each light emitting element group may be determined, and the light emitting element OFF_L1 that is turned off in response to the first bio-data may be arranged in the last row M and one of the first column 1 to the second to last column N−2, the light emitting element OFF_L2 that is turned off in response to the second bio-data may be arranged in the first row 1 and may be arranged in one column behind the column in which the light emitting element OFF_L1 that is turned off in response to the first bio-data is arranged, and the light emitting element OFF_L3 that is turned off in response to the third bio-data may be arranged in the last row M and may be arranged in one column behind the column in which the light emitting element OFF_L2 that is turned off in response to the second bio-data is arranged.

The optical communication system 1000 according to the embodiment of the present disclosure may provide a security function for each user. Because a general close circuit television camera (CCTV) including an image sensor may be used as the optical camera 210, it is important to prevent data transmitted by the transmitter 100 from being exposed to other communication devices (a smartphone, other cameras, and so on), and a method for this will be described below.

Specifically, the transmitter 100 may assign a unique key corresponding to each user. When outputting the encoded color data through a light emitting element group, the transmitter 100 may encrypt the encoded color data by using the unique key and output the encrypted color data through the light emitting element group.

Here, assuming that the data received by the optical camera 210 is referred to as r and an original signal transmitted through the light emitting element is referred to as x, an equation r=Hx+n may be established, H is an optical channel gain, and n may be noise of a light emitting element array.

When the transmitter 100 transmits encoded o instead of original x at the time of transmission, an equation r=Ho+n may be established. Here, o may be 0.00392a×cl, and a may be 0.5(x+c).

Here, cl may be uniquely set for each user, and c satisfies following Equation 1 for each user.

$$c = [ \begin{matrix}{k}_{1} & {k}_{1} & {k}_{1} & {k}_{1} \\ {k}_{2} & {k}_{2} & {k}_{2} & {k}_{2} \\ {k}_{3} & {k}_{3} & {k}_{3} & {k}_{3} \\ {k}_{4} & {k}_{4} & {k}_{4} & {k}_{4}\end{matrix} ]$$

Then, a signal received by the optical camera 210 may satisfy r=0.00196H ((x+c)cl)+n. If this is rearranged as x, x may be rearranged by Equation 2 below.

x=510((r×H⁻¹)cl⁻¹)−c Equation 2

Because the transmitter 100 and the receiver 200 know the unique key assigned to each user, even when another communication device (another optical camera, a smartphone, or so on) checks a light emitting element pattern of the light emitting element array 130, internal contents may not be known. According to an embodiment of the present disclosure, bio-data which is important for personal information security may be encrypted to be protected as described above.

After decrypting the color data encrypted with a unique key corresponding to each user, the receiver 200 may decode the color data to acquire bio-data for each type.

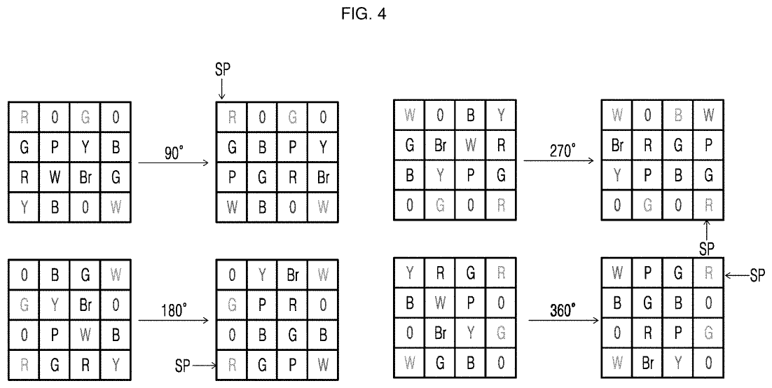

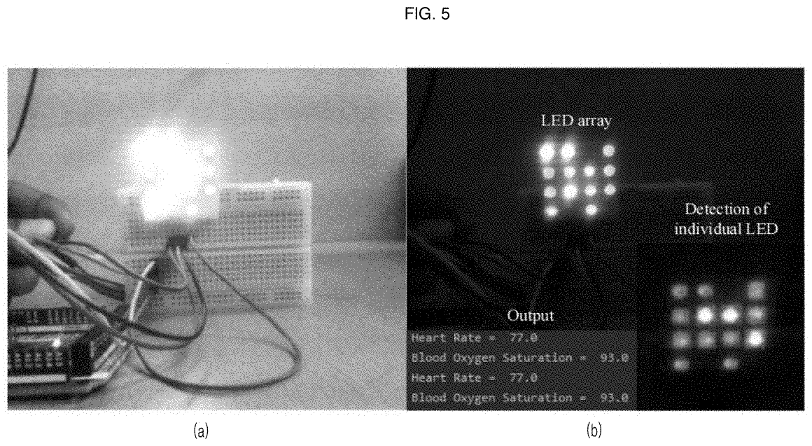

FIG. 4 illustrates diagrams for a process of recognizing rotation of a light emitting element array and decoding color data, according to an embodiment of the present disclosure.

The receiver 200 may receive successive frames through the optical camera 210, and even when the light emitting element array rotates by 90 degrees, 180 degrees, 270 degrees, and 360 degrees, an arrangement direction of the light emitting element array may be determined, and color data of each light emitting element included in the light emitting element array may be decoded.

(a) and (b) of FIG. 5 are views illustrating a method of effectively recognizing color data output from a light emitting element by adjusting a light exposure time of an optical camera, according to an embodiment of the present disclosure.

In an optional embodiment, a size of a diagnostic test site according to an embodiment of the present disclosure may be 5×4×3 meters, a frame rate of the optical camera 210 may be 30 fps, a frequency thereof may be set to 2 kHz, and an exposure time thereof may be set to 1.25 ms.

The optical camera 210 may set a background screen to be dark even when a diagnostic test site is bright as illustrated in (a) of FIG. 5. That is, the optical camera 210 may be set to have a preset exposure time capable of recognizing a light emitting element array.

In another embodiment, when the receiver 200 may set an exposure time of the optical camera 210, the optical camera 210 is controlled to a preset exposure time capable of recognizing the light emitting element array, and when the light emitting element array is recognized, the exposure time of the optical camera 210 may be gradually increased over time.

That is, when it is difficult to recognize light of the light emitting element or when it is difficult to recognize the light due to interference between light emitting elements, the receiver 200 may control the optical camera 210 as illustrated in (b) of FIG. 5 in order to recognize only the light emitting element array. When acquiring color data of the light emitting element array and the light emitting elements, the receiver 200 may gradually brighten the periphery of the light emitting element array, thereby providing easy observation of a state of a person to be diagnosed.

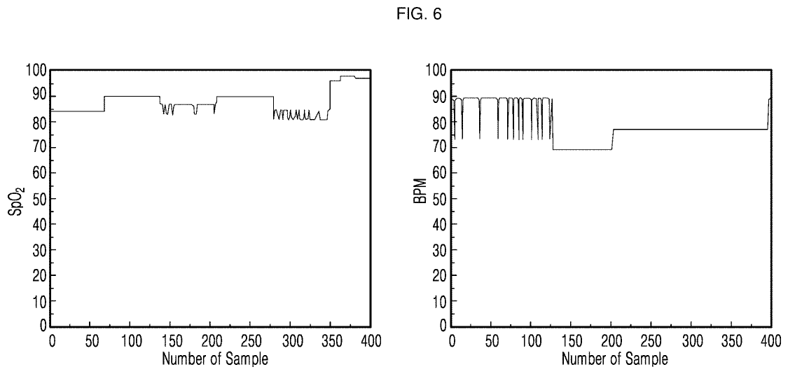

FIG. 6 illustrate diagrams for examples of values of bio-data corresponding to the number of samples in a state in which a person to be diagnosed does not move, according to an embodiment of the present disclosure.

It may be seen that the oxygen saturation (SpO₂) shows a constant pattern even when the number of samples is increased, and BPM shows a certain pattern and has a fixed value for a certain time.

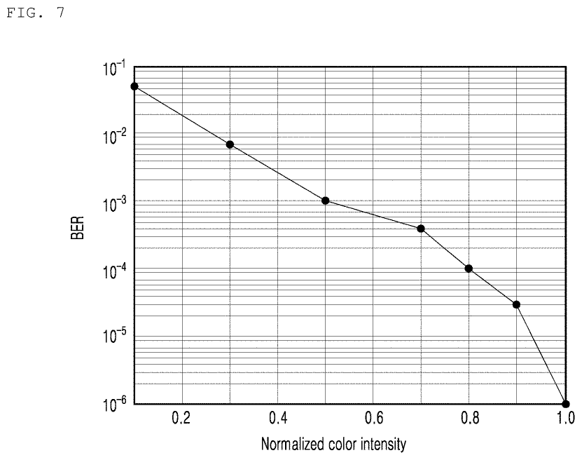

FIG. 7 is a graph illustrating a relationship between normalized color intensity and a bit error rate (BER). It may be seen that, as a normalized value increases to 1, the BER is reduced.

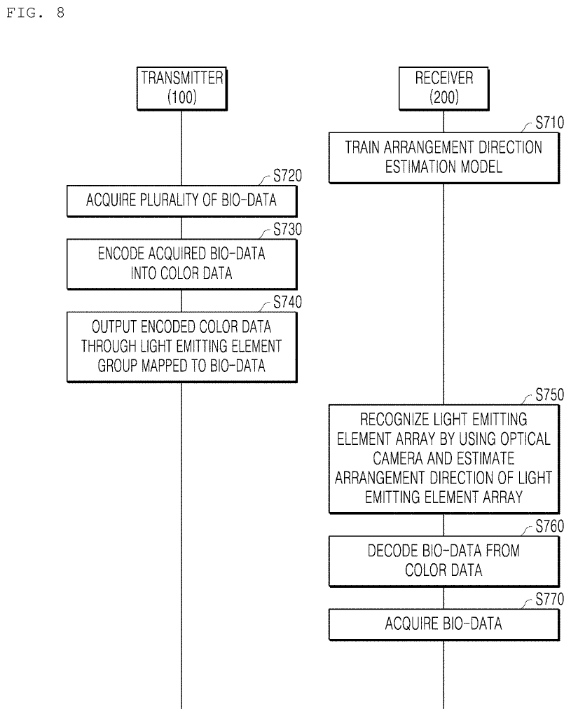

FIG. 8 is a sequence diagram illustrating a communication method of the optical communication system 1000, according to an embodiment of the present disclosure.

First, the receiver 200 trains an arrangement direction estimation model (S710).

In an optional embodiment, an external server may perform the training, and the receiver 200 may store only an arrangement direction estimation model which is previously trained without performing the training. Step S710 may be performed at any time before step S750.

The transmitter 100 acquires a plurality of bio-data (S720) and encodes the acquired bio-data into color data (S730).

The transmitter 100 outputs encoded color data through the light emitting element group mapped to the bio-data (S740).

The transmitter 100 may encrypt the encoded color data into a unique key unique to each user and transmit the encrypted color data.

Then, the receiver 200 recognizes the light emitting element array by using an optical camera and estimates an arrangement direction of the light emitting element array (S750).

Here, the receiver 200 may recognize the encoded color data of each light emitting element by using the arrangement direction estimation model which is previously trained, and further recognize color of each light emitting element by using the arrangement direction estimation model. The arrangement direction estimation model may be previously trained to recognize this.

The receiver 200 decodes the bio-data from the color data (S760) and acquires the bio-data (S770).

Here, the receiver 200 may also perform a process of decrypting the encrypted color data.

The present disclosure described above may be implemented as computer-readable codes in media in which programs are recorded. The computer-readable media include all types of recording devices in which data readable by a computer system is stored. The computer-readable media include, for example, a hard disk drive (HDD), a solid state disk (SSD), a silicon disk drive (SDD), read only memory (ROM), random access memory (RAM), compact disk (CD)-ROM, a magnetic tape, a floppy disk, an optical data storage device, and so on. In addition, the computer may include the reception controller 290 of the receiver 100.

Specifically, when the program is executed by a processor, the program may include executable instructions that cause the processor to perform an operation of recognizing a light emitting element array, an operation of recognizing an arrangement direction of light emitting elements included in the light emitting element array based on a previously stored arrangement direction estimation model based on one or more light emitting elements that are turned off, and an operation of decoding color data of a light emitting element group mapped for each type of a plurality of bio-data into corresponding bio-data to acquire the decoded bio-data.

As described above, specific embodiments of the present disclosure are described and illustrated, but the present disclosure is not limited to the embodiments described above, and those skilled in the art will understand that various modifications and variations may be made in other specific embodiments without departing from the idea and scope of the present disclosure. Accordingly, the scope of the present disclosure should not be defined by the embodiments described above and should be defined by the technical idea described in the claims.

What is claimed is:

1. A bio-information transmitting device comprising:
at least one biometric sensor configured to acquire a plurality of bio-data;
a light emitting element array; and
a transmission controller configured to map a light emitting element group included in the light emitting element array for each type of the bio-data, configured to encode the acquired bio-data into RGB-based color data, and configured to output the encoded color data through the light emitting element group,
wherein at least part of the light emitting element group is configured to turn off one light emitting element included in the light emitting element group.

2. The bio-information transmitting device of claim 1,
wherein the transmission controller assigns a unique key corresponding to each user, and
wherein, when the encoded color data is configured to output through the light emitting element group, the transmission controller encrypts the encoded color data by using the unique key and outputs the encrypted color data through the light emitting element group.

3. The bio-information transmitting device of claim 1,
wherein the plurality of bio-data includes first bio-data, second bio-data, and third bio-data,
wherein the light emitting element array has an M×N matrix (M and N are natural numbers greater than or equal to 4), and
wherein, as a position of the light emitting element to be turned off for each light emitting element group, the first bio-data is arranged in a last row (M) and one of a first column (1) to a second to last column (N−2), the second bio-data is arranged in a first row (1) and one column behind a column in which the first bio-data is arranged, and the third bio-data is arranged in the last row (M) and one column behind a column in which the second bio-data is arranged.

4. The bio-information transmitting device of claim 1,
wherein the bio-data includes infrared (IR) data, beats per minute (BPM) data, and oxygen saturation (SpO₂) data.

5. The bio-information transmitting device of claim 1,
wherein the light emitting element array is located around a user's wrist, and
wherein one biometric sensor is located at an end of a user's finger.

6. A bio-information receiving device comprising:
an optical camera configured to image a light emitting element array in an image range; and
a reception controller configured to recognize the light emitting element array and configured to recognize an arrangement direction of light emitting elements included in the light emitting element array based on an arrangement direction estimation model which is previously stored, based on one or more light emitting elements which are turned off,
wherein the reception controller is configured to decode color data of a light emitting element group mapped for each type of a plurality of bio-data into corresponding bio-data to acquire decoded bio-data.

7. The bio-information receiving device of claim 6,
wherein the arrangement direction estimation model which is previously stored is trained to estimate a number of bio-data and the arrangement direction of the light emitting elements included in the light emitting element array when receiving information on one or more light emitting element arrays prepared in advance and arrangement information of light emitting elements which are included in a light emitting element group mapped to the bio-data and are turned off.

8. The bio-information receiving device of claim 6,
wherein the reception controller is configured to decrypt color data encrypted with a unique key corresponding to each user and configured to decode the decrypted color data into the bio-data.

9. The bio-information receiving device of claim 6,
wherein the optical camera is set to have a preset exposure time required for recognizing the light emitting element array.

10. The bio-information receiving device of claim 6,
wherein the reception controller is configured to control the optical camera with a preset exposure time required for recognizing the light emitting element array, and when the light emitting element array is recognized, the exposure time of the optical camera is gradually increased over time.

11. A bio-information communication system comprising:
a transmitter; and
a receiver,
wherein the transmitter includes at least one biometric sensor configured to acquire a plurality of bio-data, a light emitting element array, and a transmission controller configured to map a light emitting element group included in the light emitting element array for each type of the bio-data, configured to encode the acquired bio-data into RGB-based color data, and configured to output the encoded color data through the light emitting element group,
wherein at least part of the light emitting element group is configured to turn off one light emitting element included in the light emitting element group,
wherein the receiver includes an optical camera configured to image a light emitting element array in an image range, and a reception controller configured to recognize the light emitting element array and configured to recognize an arrangement direction of light emitting elements included in the light emitting element array based on an arrangement direction estimation model which is previously stored, based on one or more light emitting elements which are turned off, and
wherein the reception controller is configured to decode color data of a light emitting element group mapped for each type of a plurality of bio-data into corresponding bio-data to acquire decoded bio-data.

12. The bio-information communication system of claim 11,
wherein the transmission controller assigns a unique key corresponding to each user, and
wherein, when the encoded color data is output through the light emitting element group, the transmission controller is configured to encrypt the encoded color data by using the unique key and output the encrypted color data through the light emitting element group.

13. The bio-information communication system of claim 11,
wherein the plurality of bio-data includes first bio-data, second bio-data, and third bio-data,
wherein the light emitting element array has an M×N matrix (M and N are natural numbers greater than or equal to 4), and
wherein, as a position of the light emitting element to be turned off for each light emitting element group is, the first bio-data is arranged in a last row (M) and one of a first column (1) to a second to last column (N−2), the second bio-data is arranged in a first row (1) and one column behind a column in which the first bio-data is arranged, and the third bio-data is arranged in the last row (M) and one column behind a column in which the second bio-data is arranged.

14. The bio-information communication system of claim 11,
wherein the arrangement direction estimation model which is previously stored is trained to estimate a number of bio-data and the arrangement direction of the light emitting elements included in the light emitting element array when receiving information on one or more light emitting element arrays prepared in advance and arrangement information of light emitting elements which are included in a light emitting element group mapped to the bio-data and are turned off.

15. The bio-information communication system of claim 14,
wherein the arrangement direction estimation model is trained to estimate the encoded color data included in the light emitting element group when receiving encoded color data of each of the light emitting elements included in the one or more light emitting element arrays prepared in advance.

16. The bio-information communication system of claim 11,
wherein the reception controller is configured to decrypt color data encrypted with a unique key corresponding to each user and configured to decode the decrypted color data into the bio-data.

17. The bio-information communication system of claim 11,
wherein the optical camera is set to have a preset exposure time required for recognizing the light emitting element array.

18. The bio-information communication system of claim 11,
wherein the reception controller is configured to control the optical camera with a preset exposure time required for recognizing the light emitting element array, and when the light emitting element array is recognized, the exposure time of the optical camera is gradually increased over time.
